(19) United States
(12) Patent Application Publication
(10) Pub. No.: US 2018/0339053 A1
(43) Pub. Date: Nov. 29, 2018

(54) USE OF 1,2-DICARBOXYLIC ACID MONOAMIDE POLYMER AS SYNERGIST FOR CHEMOTHERAPY

(71) Applicant: Nankai University, Tianjin (CN)

(72) Inventors: Husheng YAN, Tianjin (CN); Jun CAO, Tianjin (CN)

(21) Appl. No.: 15/987,005

(22) Filed: May 23, 2018

(30) Foreign Application Priority Data
May 26, 2017 (CN) 201710402913.X

(51) Int. Cl. A61K 47/34; A61K 47/32; A61K 31/704; A61P 35/00; A61K 9/00

(52) U.S. Cl. CPC A61K 47/34; A61K 47/32; A61K 9/0019; A61P 35/00; A61K 31/704

(57) ABSTRACT

A use of a polymer as a synergist for a chemotherapeutic drug, wherein the polymer is a polymer having a 1,2-dicarboxylic acid monoamide structure, and after the polymer, a chemotherapeutic drug and a cancer cell are co-incubated in a slightly acidic environment which simulates a tumor tissue, a survival rate of the cancer cell is lower than that of a cancer cell incubated under the same conditions without the polymer. The polymer and the chemotherapeutic drug are injected together into a tumor-bearing animal body, the tumor-growth inhibiting effect thereof is better than that with injection of the same dosage of the chemotherapeutic drug alone.

This application claims priority to Chinese application number 201710402913.X, filed 26 May 2017, with a title of USE OF 1,2-DICARBOXYLIC ACID MONOAMIDE POLYMER AS SYNERGIST FOR CHEMOTHERAPY. The above-mentioned patent application is incorporated herein by reference in its entirety.

### TECHNICAL FIELD

The present invention relates to the field of a synergist for an anti-tumor drug, and in particular to use of a charge-transferable polymer with a 1,2-dicarboxylic acid monoamide structure for increasing the uptake of a chemotherapeutic drug by a tumor cell, improving an anti-tumor effect and overcoming the drug resistance of the tumor cell.

### BACKGROUND

A chemotherapeutic therapy (chemotherapy) realizes a therapeutic purpose by using a chemotherapeutic drug to kill cancer cells. As one of the most effective means for treating cancer currently, chemotherapy is not only a palliative therapy for relieving cancer or an auxiliary method for surgery and radiation therapy, but also can cure some cancers sensitive to the chemotherapeutic drug such as leukemia, lymphoma, chorionic epithelioma and the like malignant tumors. However, the chemotherapeutic drug often has large toxicity and strong side effects, and also causes damage to normal tissue cells while killing cancer cells, leading to myelosuppression, gastrointestinal toxicity, skin toxicity, allergy, and the like adverse effects. Some anti-cancer drugs further cause adverse effects in the nervous system, respiratory system, heart, liver, urinary system and the like. A traditional administration method mainly includes oral administration and injection. There is no selection for distribution of drugs in the body. The drug concentration at the tumor site is low, and thus the pharmaceutical effect is low. However, increasing the uptake amount of the drug will increase the side effects of the drug as well as the toxicity. Furthermore, it has been reported that during the chemotherapy, more than 90% of patients develop intrinsic or acquired resistance to the chemotherapeutic drug (Baguley B C. Multidrug resistance in cancer.2010: 1-14). Studies have shown that a cancer cell that develops resistance to certain chemotherapeutic drugs also develops resistance to other anti-cancer drugs with different structures and action mechanisms. This multidrug resistance has become a major cause of clinical chemotherapy failure (Vtorushin S V, et al. The phenomenon of multi-drug resistance in the treatment of malignant tumors, Exp Oncol, 2014, 36(3): 144-56).

A nano drug carrier system achieves specific treatment of cancer through targeted delivery and reversal of multi-drug resistance by combining an anti-cancer drug with a nano-scale material. The particle size of the nano drug carrier is generally between several nanometers to several hundred nanometers, and has advantages of a small particle size, a strong specific surface area effect, and high dispersity. The vascular wall of a normal tissue has tight gaps and a complete structure, while a solid tumor tissue is rich of blood vessels, the vascular wall has larger gaps and the integrity of the vascular wall structure is poor, such that the blood vessel of the tumor tissue has higher permeability to macromolecule substances than that of the normal blood vessel, and additionally since the tumor tissue is lack of lymphatic circulation, the contact time and contact area of the macromolecules and the drug at a specific site are increased. This high permeability and retention of macromolecules in a tumor tissue is referred to as enhanced permeability and retention (EPR) (Maeda H, Nakamura H, Fang J. The EPR effect for macromolecular drug delivery to solid tumors: Improvement of tumor uptake, lowering of systemic toxicity, and distinct tumor imaging in vivo, Advanced drug delivery reviews, 2013, 65(1): 71-79). Based on the EPR effect, nanoparticles can be selectively distributed at the tumor site, which enhances the uptake and availability of the drug by an organism.

Based on the various advantages of the nano-drug carrier system, it has stimulated a wide range of interests of scientific researchers. In the past ten years, a lot of researches have been carried out around the nano-drug carrier system, and many scientific papers have been published therefor. However, there are few nano-drugs which enter the clinical application period based on the nano-drug carrier system, and even for several nano-drugs which have already clinically applied, they only have slightly-reduced side effects and have no significant increase in the pharmaceutical effect compared with a traditional chemotherapeutic drug. The first reason is that the drug delivery efficiency of this system is too low, and the amount of the drug reaching the tumor site is less than 1% of the administration dosage (Wilhelm S, et al. Analysis of nanoparticle delivery to tumours, Nature Reviews Materials, 2016, 1: 16014). The reason for the low delivery efficiency of the drug is mainly caused by any one or more of the following factors: (1) the carrier has low drug loading capacity; (2) the nano-particles have short blood circulation time; (3) the loaded drug in the blood is released in advance; and (4) the drug entered the tumor tissue or the cancer cell cannot be released quickly and completely. The second reason is that, because of the low permeability of nanoparticles at the tumor site, the tumor tissue has a dense extracellular matrix and intratumoral hyperosmolality caused by lack of lymphatic drainage, such that the fluid pressure in the tumor tissue space is usually 10-40 times that of a normal tissue, thereby resulting in a gradient difference and heterogeneous flow which affect the distribution and penetration of macromolecular drug carrier particles (Swartz M A, Lund A W. Lymphatic and interstitial flow in the tumour microenvironment: linking mechanobiology with immunity, Nature Reviews Cancer, 2012, 12(3): 210-219). Therefore, it is difficult for the nano-particles reaching the tumor site through blood circulation to penetrate deep into the tumor tissue to kill the tumor cells. Studies have demonstrated that, as compared with nano-particles with large particle sizes which can only penetrate into the tumor tissue surrounding the blood vessel, the nano-particles with small particle sizes can penetrate more deep into the tumor to play a role (Albanese A, et al. Tumour-on-a-chip provides an optical window into nanoparticle tissue transport, Nature communications, 2013, 4), wherein particles having a particle size less than 12 nm has a great penetration effect (Chauhan V P, et al. Normalization of tumour blood vessels improves the delivery of nanomedicines in a size-dependent manner, Nature nanotechnology, 2012, 7(6): 383-388).

The surface properties of nano-particles affect their circulation and distribution and interaction with cells in vivo. Nanoparticles with positively-charged or hydrophobic surfaces can interact strongly with negatively-charged tumor cell membranes to enhance endocytosis. However, positively-charged nano-particles also interact with plasma proteins and phagocyte membranes in vivo, such that the nano-particles are absorbed by the proteins and cleared by phagocytic cells, which reduces residence time of these nanoparticles in vivo, and meanwhile the positively-charged nano-particles are cytotoxic and have an effect on normal tissues. Nano-particles with negatively-charged surfaces significantly increase their circulation time in vivo, but the interaction of them with tumor cells is also weakened accordingly, and the endocytosis of cells is decreased, which affects the therapeutic effect of drugs. In order to solve the aforementioned contradiction, a nano-particle system with transferable surface charges has been developed, which is characterized in that the particle has negative charges on the surface thereof in the blood circulation and has great in vivo circulation properties, and at the tumor site the charges on the surface of the nano-particle becomes positive. The particle then interacts strongly with cell membranes to increase endocytosis, increase the uptake of anti-tumor drugs by a tumor cell, and enhance the drug therapeutic effect. An amino-containing polycation can interact strongly with negatively-charged cell membranes and generate a proton sponge effect, and thus is widely used in the research of drug delivery and gene delivery. By utilizing a nano-particle drug carrier system with a substituted maleic acid monoamide structure or a substituted succinic acid monoamide structure, when nano-carriers accumulate at the tumor site via the EPR effect, the amido bond is hydrolyzed to expose the amino group due to the slightly acidic environment of the tumor, such that the nano-particles are positively charged and easily enter the cancer cells to further release the carried drug (Zhou Z, et al. Linear polyethyleneimine-based charge-reversal nanoparticles for nuclear-targeted drug delivery. Journal of Materials Chemistry, 2011, 21(47): 19114-19123; Tang S, et al. Dual pH-sensitive micelles with charge-switch for controlling cellular uptake and drug release to treat metastatic breast cancer. Biomaterials, 2017, 114: 44-53; Zhou Z, et al. Molecularly precise dendrimer-drug conjugates with tunable drug release for cancer therapy. Angew. Chem. Int. Ed. 2014, 53, 10949-10955). However, these systems still cannot overcome inherent disadvantages of the nano-particle drug carrier system.

Another report of promoting the entry of chemotherapeutic drugs into cancer cells is that, the manner of administrating a physical mixture of a tumor-penetrating peptide iRGD and an anti-cancer drug effectively improves the anti-cancer effect, and it has a higher inhibition effect on tumor cells in vitro cell experiments and animal experiments as compared with nano-particle carriers which are bonded to and loaded with anti-cancer drugs, and meanwhile since it can reduce the use dosage of anti-cancer drugs, the side effects of anti-cancer drugs can be further reduced. (Sugahara K N, et al. Coadministration of a tumor-penetrating peptide enhances the efficacy of cancer drugs. Science, 2010, 328(5981): 1031-1035). This manner utilizes the action of a tumor-penetrating peptide to promote the uptake of anti-tumor drugs by cells, and therefore has potential research values. However, the polypeptide is easily degraded in vivo, and such a shortcoming limits the practical application value of this method. To overcome this shortcoming, nanodiamonds and nanoplatinum are used as accelerants for prompting the chemotherapeutic drugs to enter cells (Ghoneum A, et al. Nano-hole induction by nanodiamond and nanoplatinum liquid, DPV576, reverses multidrug resistance in human myeloid leukemia (HL60/AR). International Journal of Nanomedicine 2013, 8, 2567-2573). However, this accelerating agent for promoting the entry of chemotherapeutic drugs into cells can only work at a very high concentration, and therefore has little practical application value.

### SUMMARY

It is reported in a literature that positively-charged polymers and nano-particles have a strong performance of increasing the permeability of cell membranes (Leroueil P R, et al. Nanoparticle interaction with biological membranes: Does nanotechnology present a janus face? Acc. Chem. Res. 2007, 40, 335-342). The present invention allows more chemotherapeutic drugs to enter cancer cells by increasing the permeability of cancer cell membranes by means of positively-charged polymers or nano-particles. However, instead of loading a chemotherapeutic drug into a carrier as recited in the literature, the entry of a chemotherapeutic drug into a cancer cell is promoted by administrating a mixture of the chemotherapeutic drug and a chemotherapy accelerator, which overcomes the inherent disadvantages of the aforementioned nano-drug carrier system. However, positively-charged polymers and nano-particles are highly toxic and can be easily cleared in the body. The strategy of the present invention is to use a polymer or a nano-particle containing a primary or secondary amine group to form a monoamide structure with a substituted butenedioic acid or a substituted succinic acid, and the formed amido bond is relatively stable under a normal physiological environment (pH 7.4). That is, polymers or nano-particles having such amido bonds are negatively charged under the normal physiological environment and thus have low toxicity and long blood circulation time. After such polymers or nano-particles enter a tumor tissue, the amido bond is hydrolyzed due to a slightly acidic environment of the tumor tissue, such that the polymers or nano-particles release an amine group to make polymers or nano-particles be positively charged, thereby promoting more co-administrated chemotherapeutic drugs to enter cancer cells. That is, such polymers or nano-particles are a synergist for traditional chemotherapy.

The polymer having a 1,2-dicarboxylic acid monoamide structure adopted in the present invention can be obtained through a reaction between a polymer or nano-particle having a primary and/or secondary amine group and an anhydride of 1,2-dicarboxylic acid. A schematic diagram of the reaction between a polymer or nano-particle having a primary amine group and 2,3-dimethylmaleic anhydride is as follows:

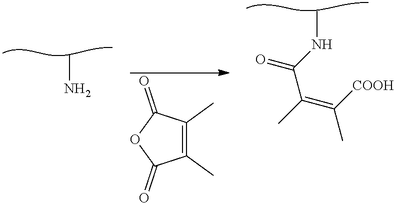

The polymer having an amine group may include, but not limited to: polyallylamine, poly(vinyl amine), linear polyethyleneimine (also known as polymine), branched polyethyleneimine, high-molecular-weight polyethyleneimine obtained by crosslinking of low-molecular-weight polyethylenimine, polylysine, polyarginine, and a hyperbranched polymer containing a primary or secondary amino group, wherein the molecular weight of the polymer ranges from 1,000 to 1,000,000 Da. The following reaction formula is a schematic view of preparing a hyperbranched polyglycerol having a primary amino group as an end group based on hyperbranched polyglycerol (for the preparation method, see Kainthan R K, et al. Synthesis, characterization, and viscoelastic properties of high molecular weight hyperbranched polyglycerols. Macromolecules 2006, 39, 7708-7717);

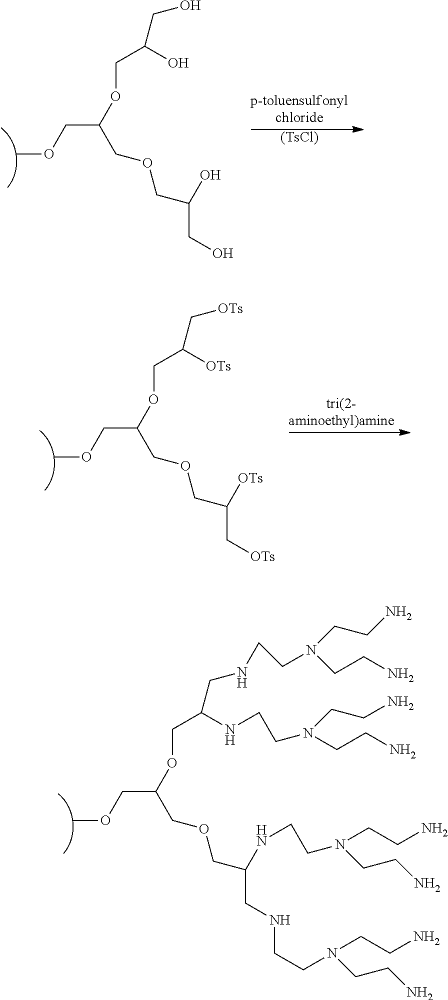 [SMILES: COCC(COCC(O)CO)OCC(O)CO ; "(TsCl)" ; COCC(COCC(COS(=O)(=O)c1ccc(C)cc1)OS(=O)(=O)c1ccc(C)cc1)OCC(COS(=O)(=O)c1ccc(C)cc1)OS(=O)(=O)c1ccc(C)cc1 ; COCC(COCC(CNCCN(CCN)CCN)NCCN(CCN)CCN)OCC(CNCCN(CCN)CCN)NCCN(CCN)CCN]

The nanoparticle having the amine group includes, but not limited to, dendritic polylysine, dendritic polyamidoamine, dendritic polypropylenimine, polylysine, and the like. The generation number of the dendritic polymer is from 2 to 10. The 1,2-dicarboxylic acid that forms the monoamide with the amine-containing polymer or nano-particle includes: maleic anhydride, 1-methylmaleic anhydride, 2,3-dimethylmaleic anhydride, cyclohexene-1,2-dicarboxylic acid, cyclohexane-1,2-dicarboxylic acid, aconitic acid, and the like. The schematic cross-linking diagram of obtaining the high-molecular-weight polyethyleneimine by crosslinking the low-molecular-weight polyethyleneimine is as follows:

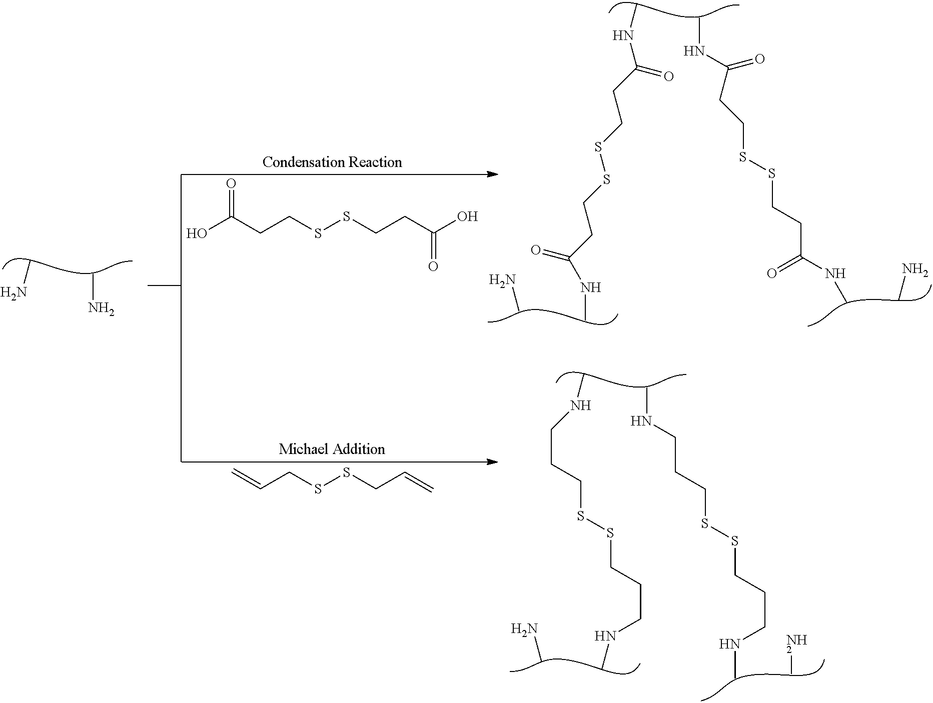

After said polymer having the 1,2-dicarboxylic acid monoamide structure has been cultivated in a tumor-simulating environment (e.g., pH 6.5) which is slightly more acidic than the normal physiological environment for a certain period of time, the amido bond is hydrolyzed such that the polymer releases the amino group and thus the polymer is positively charged. The positively-charged polymer increases the permeability of the cell membrane and thus makes it easier for the anti-cancer drug to enter the cancer cell, thereby increasing the anti-cancer effect of the anti-cancer drug. The synergistic ability of the drug of the present invention is tested via an in vitro cell experiment and a tumor-bearing animal experiment. In a toxicity experiment using a non-resistant human hepatocellular carcinoma HepG2 cell as a model cell, as compared with the use of an anti-cancer drug alone, the polymer having the 1,2-dicarboxylic acid monoamide structure increases the cancer cell lethality of the anti-cancer drug by 16-42 percentage points, and the anti-cancer effect of the anti-cancer drug is increased by 2-4 times. For a doxorubicin-resistant human hepatocellular carcinoma HepG2/ADR cell, the polymer having the 1,2-dicarboxylic acid monoamide structure increases the cancer cell lethality of the anti-cancer drug by 20-60 percentage points, and especially has a very significant improving effect on the pharmaceutical effect of doxorubicin as it increases the pharmaceutical effect of doxorubicin by three orders of magnitude, reversing the drug resistance of the cell. It is shown by the results of a further tumor-bearing animal experiment that, as compared with the use of the anti-cancer drug alone, after the use of the synergist as described by the present invention, the tumor weight is reduced by 60% to 80%, and the in vivo anti-cancer effect of the anti-cancer drug is improved. The beneficial effects of the present invention are: the preparation method of the synergist is simple; there are a wide variety of applicable raw materials; the organic solvent is not used and thus is environmentally friendly and safe; the product is easy to store and is convenient for transportation; the usage mode is simple and convenient; and it has an ideal synergistic effect on various kinds of chemotherapeutic drugs.

The following describes the present invention in detail with reference to the accompanying drawings and specific embodiments.

### BRIEF DESCRIPTION OF DRAWINGS

FIG. 1 shows the cytotoxicity experiment results of a polyallylamine-cyclohexene-1,2-dicarboxylic acid modifier (PAA200K-TPA) with a molecular weight of 200,000 on a non-resistant human hepatocellular carcinoma HepG2 cell;

FIG. 2 shows the cytotoxicity experiment results of a polyallylamine-cyclohexene-1,2-dicarboxylic acid modifier (PAA200K-TPA) with a molecular weight of 200,000 on a doxorubicin-resistant human hepatocellular carcinoma HepG2/ADR cell;

FIG. 3 shows the cytotoxicity experiment results of a 3rd-generation dendritic polypropyleneimine-2,3-dimethyl maleic anhydride modifier (G3 PPI-DMA) on a non-resistant human hepatocellular carcinoma HepG2 cell;

FIG. 4 shows the cytotoxicity experiment results of an 8th-generation dendritic polyamidoamine-2,3-dimethyl maleic anhydride modifier (G8 PAMAM-DMA) on the non-resistant human hepatocellular carcinoma HepG2 cell;

FIG. 5 shows the cytotoxicity experiment results of an 8th-generation dendritic polyamidoamine-2,3-dimethyl maleic anhydride modifier (G8 PAMAM-DMA) on the doxorubicin-resistant human hepatocellular carcinoma HepG2/ADR cell;

FIG. 6 shows the results of an in vivo experiment in which the 8th-generation dendritic polyamidoamine-2,3-dimethyl maleic anhydride modifier (G8 PAMAM-DMA) promotes the anti-tumor effect of doxorubicin (DOX);

FIG. 7 shows the results of an in vivo experiment in which a linear polyethyleneimine-cyclohexene-1,2-dicarboxylic acid modifier with a molecular weight of 10,000 (LPEI_{10K}-TPA) promotes the anti-tumor effect of doxorubicin; and

FIG. 8 shows the results of an in-vivo experiment in which a branched polyethyleneimine-1-methyl maleic anhydride modifier with a molecular weight of 25,000 (BPEI_{25K}-CA) promotes the anti-tumor effect of doxorubicin.

### DETAILED DESCRIPTION

The present invention will be further illustrated by the following embodiments, only for the purpose of better understanding the content of the present invention. It should be understood that the content of the present invention should not be limited to the scope of the embodiments, and the claimed scope of the present invention is determined by the scope of the appended claims.

### Embodiment 1

24 mg of 1,1,1-trimethylolpropane was weighed, added into 20 μL of a 25% solution of potassium methoxide in methanol, and distilled under reduced pressure to remove excess methanol. 10 mL of glycidol was slowly added into the trimethylolpropane under protection of nitrogen, where the dropwise adding time was no less than 12 hours and the reaction was conducted at 95° C. After completion of the reaction, the product was dissolved in methanol, passed through a cationic resin to remove potassium ions, and precipitated twice in diethyl ether to obtain a hyperbranched polyglycerol having a weight-average molecular weight of 50,000 as measured by gel permeation chromatography. 1.0 g of the hyperbranched polyglycerol was dissolved in 15 mL of tetrahydrofuran, added with 4.3 g of p-toluenesulfonyl chloride and 4.5 mL of triethylamine, reacted at room temperature for 12 hours, filtered to remove triethylamine salts, and precipitated in diethyl ether to obtain a p-toluenesulfonyl modified hyperbranched polyglycerol. It was dissolved in 15 mL dioxane, added with 4.6 mL of tris(2-aminoethyl)amine, reacted at room temperature for 24 hours, then distilled under reduced pressure to remove the dioxane, added with a small amount of methanol, and precipitated twice in diethyl ether to obtain a hyperbranched polyglycerol having a terminal amino group with a molecular weight of 50,000 (BPG_{50K}-NH₂).

### Embodiment 2

0.6 g of an 8th-generation dendritic polyamidoamine (G8 PAMAM) was weighed and dissolved in 10 mL water, distributed uniformly in an ice-water bath by stirring, and then added with 0.4 g of 2,3-dimethyl maleic anhydride (DMA) in multiple times. Upon the addition of DMA, the pH of the system was significantly decreased, and sodium hydroxide solution was added to keep the pH of the reaction solution between 8 and 9. After the addition of DMA was completed, the reaction was continued at room temperature for 24 hours. After the reaction was completed, the reaction solution was added to a dialysis bag, dialyzed in a sodium hydroxide solution having a pH of 10 for 72 hours, and freeze-dried to obtain an 8th-generation dendritic polyamidoamine-2,3-dimethylmaleic anhydride modifier (G8 PAMAM-DMA).

### Embodiment 3

0.6 g of a linear polyethyleneimine with a molecular weight of 10,000(LPEI_{10K}) was dissolved in 15 mL water, distributed uniformly in an ice-water bath by stirring, and then added with 1.8 g of cyclohexene-1,2-dicarboxylic anhydride (TPA) in multiple times. Upon the addition of TPA, the pH of the system was significantly decreased, and sodium hydroxide solution was added to keep the pH of the reaction solution between 8 and 9. After the addition of TPA was completed, the reaction was continued at room temperature for 24 hours. After the reaction was completed, the reaction solution was added to a dialysis bag and dialyzed in a sodium hydroxide solution having a pH of 10 for 72 hours, and freeze-dried to obtain a linear polyethyleneimine-cyclohexene-1,2-dicarboxylic acid modifier having a molecular weight of 10,000 (LPEI_{10K}-TPA).

### Embodiment 4

A suspension of HepG2 cells diluted in 100 μL of a complete medium (a RPMI-1640 medium containing 10% fetal bovine serum and 1% penneillin/streptomycin) was added into a 96-well plate at a concentration of 5×10³ cells per well, cultivated at 37 degrees Celsius for 24 hours. Thereafter, the cell culture medium was aspirated, and the cells were washed twice with a PBS solution, then added respectively with a 100 μL mixed solution of a polyallylamine-maleic anhydride modifier with a molecular weight of 10,000 (PAA_{10K}-MA) and a concentration of 25 μg/mL and different kinds of chemotherapeutic drugs (4 μg/mL doxorubicin or 5 μg/mL taxol) as diluted in a serum-free medium at different pH (6.5, 7.4), and as a control 25 μg/mL PAA_{10K}-MA solution or chemotherapeutic drugs (4 μg/mL doxorubicin or 5 μg/mL taxol) were respectively added into other wells and cultured for 3 hours. The solution was then aspirated, added with 100 μL of fresh complete medium and incubated continually for 24 hours. The culture medium was aspirated, added with 80 μL PBS solution and 20 μL cell proliferation reagent MTS, incubated at 37 degrees Celsius for 1 hour, and then measured with a microplate reader at 490 nm for absorbance. The survival rate of non-resistant cells was calculated according to the equation ODₛₐₘₚₗₑ/OD_{blank}×100%, as shown in Table 1.

The polymers represented by the abbreviations in the table are as follows: PAA_{10K}-MA: a polyallylamine-maleic anhydride modifier with a molecular weight of 10,000; PAA_{15K}-DMA: a polyallylamine-2,3-dimethyl maleic anhydride modifier with a molecular weight of 15,000; PAA_{200K}-TPA: a polyallylamine-cyclohexene-1,2-dicarboxylic acid modifier with a molecular weight of 200,000; LPEI_{10K}-DMA: a linear polyethylenimine-2,3-dimethyl maleic anhydride modifier with a molecular weight of 10,000; LPEI_{10K}-TPA: a linear polyethylenimine-cyclohexene-1,2-dicarboxylic acid modifier with a molecular weight of 10,000; BBPEI_{25K}-CA: a branched polyethyleneimine-1-methyl maleic anhydride modifier with a molecular weight of 25,000; G3 PPI-AA: a 3rd-generation dendritic polypropyleneimine-3-carboxy-2-pentenedioic acid modifier; G3 PPI-DMA: a 3rd-generation dendritic polypropyleneimine-2,3-dimethyl maleic anhydride modifier; LPLys_{2K}-MA: a linear polylysine-maleic anhydride modifier with a molecular weight of 2,000; BBPLys_{2K}-TPA: a branched polylysine-cyclohexene-1,2-dicarboxylic acid modifier having a molecular weight of 2,000; G5 DPlys-DMA: a 5th-generation dendritic polylysine-2,3-dimethyl maleic anhydride modifier; G3 PAMAM-CA: a 3rd-generation dendritic polyamidoamine-1-methyl maleic anhydride modifier; G8 PAMAM-DMA: a 8th-generation dendritic polyamidoamine-2,3-dimethyl maleic anhydride modifier; and BBPG_{50K}-DMA: a hyperbranched polyglycerol-2,3-dimethyl maleic anhydride modifier with a molecular weight of 50,000.

TABLE 1
Cytotoxicity experiment of a polymer having a 1,2-dicarboxylic acid monoamide structure and an
anti-cancer drug on HepG2 cells

| | pH = 6.5 Cell Viability (%) | | | | | pH = 7.4 Cell Viability (%) | | | | |
|---|---|---|---|---|---|---|---|---|---|---|
| | Polymer | | Polymer | | | Polymer | | Polymer | | |
| | + | | + | | | + | | + | | |
| | Doxorubicin | Doxorubicin | Taxol | Taxol | Polymer | Doxorubicin | Doxorubicin | Taxol | Taxol | Polymer |
| PAA_{10K}-MA | 40.64 | 76.97 | 45.25 | 80.34 | 84.99 | 55.48 | 72.89 | 70.49 | 83.12 | 96.39 |
| PAA_{15K}-DMA | 33.68 | 76.97 | 33.28 | 80.34 | 72.48 | 44.92 | 72.89 | 49.81 | 83.12 | 83.18 |
| PAA_{200K}-TPA | 26.83 | 76.97 | 29.92 | 80.34 | 69.52 | 37.18 | 72.89 | 42.01 | 83.12 | 77.91 |
| LPEI_{10K}-DMA | 19.72 | 78.59 | 24.87 | 78.66 | 82.71 | 34.82 | 70.75 | 42.48 | 82.38 | 93.22 |
| LPEI_{10K}-TPA | 29.81 | 78.59 | 42.98 | 78.66 | 82.22 | 62.28 | 70.75 | 71.07 | 82.38 | 88.71 |
| BPEI_{25K}-CA | 13.29 | 78.59 | 15.02 | 78.66 | 73.92 | 53.65 | 70.75 | 65.36 | 82.38 | 86.12 |
| G3 PPI-AA | 35.85 | 78.59 | 32.14 | 78.66 | 80.66 | 55.19 | 70.75 | 52.59 | 82.38 | 91.39 |
| G3 PPI-DMA | 22.93 | 78.59 | 18.36 | 78.66 | 62.09 | 53.84 | 70.75 | 47.60 | 82.38 | 77.14 |
| LPLys_{2K}-MA | 31.46 | 77.13 | 34.62 | 76.08 | 77.73 | 42.84 | 73.82 | 50.61 | 80.61 | 90.72 |
| BPLys_{2K}-TPA | 27.56 | 77.13 | 26.71 | 76.08 | 73.98 | 36.84 | 73.82 | 40.12 | 80.61 | 85.83 |
| G5 DPlys-DMA | 23.58 | 77.13 | 33.25 | 76.08 | 73.05 | 43.52 | 73.82 | 61.34 | 80.61 | 87.73 |
| G3 PAMAM-CA | 42.10 | 77.13 | 53.65 | 76.08 | 87.02 | 56.48 | 73.82 | 68.49 | 80.61 | 93.12 |
| G8 PAMAM-DMA | 13.65 | 77.13 | 15.76 | 76.08 | 64.38 | 30.46 | 73.82 | 39.27 | 80.61 | 78.55 |
| BPG_{50K}-DMA | 36.28 | 77.13 | 30.19 | 76.08 | 78.82 | 62.63 | 73.82 | 59.75 | 80.61 | 86.03 |

### Embodiment 5

A suspension of HepG2/ADR cells diluted in 100 μL of a complete medium (a RPMI-1640 medium containing 10% fetal bovine serum and 1% penneillin/streptomycin) was added into a 96-well plate at a concentration of 5×10³ cells per well, cultivated at 37 degrees Celsius for 24 hours. Thereafter, the cell culture medium was aspirated, and the cells were washed twice with a PBS solution, then added respectively with a 100 μL mixed solution of PAA_{10K}-MA at a concentration of 25 μg/mL and different kinds of chemotherapeutic drugs (4 μg/mL doxorubicin or 5 μg/mL taxol) as diluted in a serum-free medium at different pH (6.5, 7.4), and as a control 25 μg/mL PAA_{10K}-MA solution or chemotherapeutic drugs (4 μg/mL doxorubicin or 5 μg/mL taxol) were respectively added into other wells and cultured for 3 hours. The solution was then aspirated, added with 100 μL of fresh complete medium and incubated continually for 24 hours. The culture medium was aspirated, added with 80 μL PBS solution and 20 μL cell proliferation reagent MTS, incubated at 37 degrees Celsius for 2 hours, and then measured with a microplate reader at 490 nm for absorbance. The survival rate of multidrug-resistant cells was calculated according to the equation ODₛₐₘₚₗₑ/OD_{blank}×100%, as shown in Table 2.

For the abbreviations of polymers in the table, reference is made to Example 4.

TABLE 2
Cytotoxicity experiment of a polymer haying a 1,2-dicarboxylic acid monoamide structure and an
anti-cancer drug on HepG2/ADR cells

| | pH = 6.5 Cell Viability (%) | | | | | pH = 7.4 Cell Viability (%) | | | | |
|---|---|---|---|---|---|---|---|---|---|---|
| | Polymer | | Polymer | | | Polymer | | Polymer | | |
| | + | | + | | | + | | + | | |
| | Doxorubicin | Doxorubicin | Taxol | Taxol | Polymer | Doxorubicin | Doxorubicin | Taxol | Taxol | Polymer |
| PAA_{10K}-MA | 50.38 | 100.38 | 60.75 | 84.47 | 88.43 | 66.75 | 99.23 | 70.85 | 89.17 | 96.31 |
| PAA_{15K}-DMA | 47.74 | 100.38 | 48.93 | 84.47 | 86.74 | 59.85 | 99.23 | 72.85 | 89.17 | 97.64 |
| PAA_{200K}-TPA | 38.73 | 100.38 | 32.98 | 84.47 | 74.03 | 50.87 | 99.23 | 47.27 | 89.17 | 93.37 |
| LPEI_{10K}-DMA | 30.40 | 100.98 | 27.59 | 86.09 | 84.11 | 39.02 | 98.74 | 47.88 | 91.68 | 90.81 |
| LPEI_{10K}-TPA | 31.66 | 100.98 | 30.71 | 86.09 | 87.33 | 48.81 | 98.74 | 55.93 | 91.68 | 82.01 |
| BPEI_{25K}-CA | 21.82 | 100.98 | 24.66 | 86.09 | 80.24 | 52.62 | 98.74 | 69.73 | 91.68 | 93.39 |
| G3 PPI-AA | 41.86 | 100.98 | 35.77 | 86.09 | 85.12 | 66.41 | 98.74 | 70.11 | 91.68 | 95.32 |
| G3 PPI-DMA | 26.06 | 100.98 | 30.52 | 86.09 | 83.84 | 64.78 | 98.74 | 66.52 | 91.68 | 96.85 |
| LPlys_{2K}-MA | 40.57 | 99.98 | 43.99 | 85.43 | 85.87 | 65.33 | 98.83 | 64.07 | 91.22 | 92.61 |
| BPLys_{2K}-TPA | 38.13 | 99.98 | 42.43 | 85.43 | 83.75 | 55.25 | 98.83 | 69.02 | 91.22 | 94.84 |
| G5 DPlys-DMA | 28.75 | 99.98 | 36.44 | 85.43 | 85.63 | 50.63 | 98.83 | 71.22 | 91.22 | 91.53 |
| G3 PAMAM-CA | 53.85 | 99.98 | 57.11 | 85.43 | 93.91 | 68.55 | 98.83 | 73.28 | 91.22 | 97.76 |
| G8 PAMAM-DMA | 22.77 | 99.98 | 20.08 | 85.43 | 70.41 | 43.71 | 98.83 | 50.41 | 91.22 | 89.73 |
| BPG_{50K}-DMA | 42.72 | 99.98 | 38.77 | 85.43 | 83.17 | 66.35 | 98.83 | 63.76 | 91.22 | 95.32 |

### Embodiment 6

This embodiment is an in vitro cell experiment of the effect of a polyallylamine-2,3-dimethyl maleic anhydride modifier with a molecular weight of 15,000 (PAA_{15K}-DMA) for promoting the toxicity of different chemotherapeutic drugs on non-resistant HepG2 cells. According to the method described in Embodiment 4, the concentration of PAA_{15K}-DMA was 25 μg/mL, and the obtained data was shown in Table 1.

### Embodiment 7

This embodiment is an in vitro cell experiment of the effect of PAA_{15K}-DMA for promoting the toxicity of different chemotherapeutic drugs on multidrug-resistant cells HepG2/ADR. According to the method described in Embodiment 5, the concentration of PAA_{15K}-DMA was 25 μg/mL, and the obtained data was shown in Table 2.

### Embodiment 8

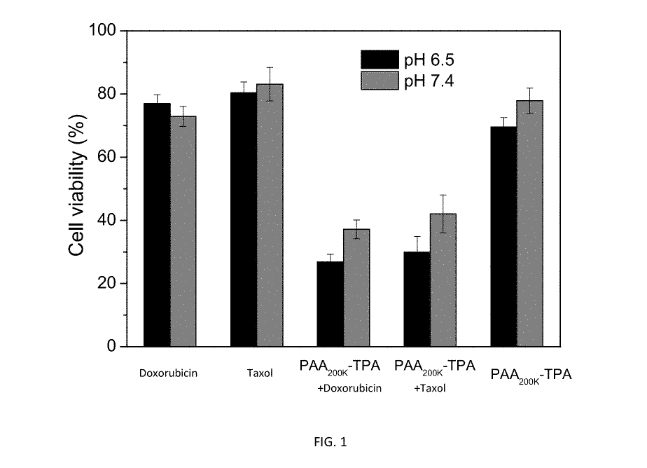

This embodiment is an in vitro cell experiment of the effect of a polyallylamine-cyclohexene-1,2-dicarboxylic acid modifier with a molecular weight of 200,000 (PAA_{200K}-TPA) for promoting the toxicity of different chemotherapeutic drugs on non-resistant HepG2 cells. According to the method described in Embodiment 4, the concentration of PAA_{200K}-TPA was 25 μg/mL, and the obtained data was shown in Table 1 and FIG. 1.

### Embodiment 9

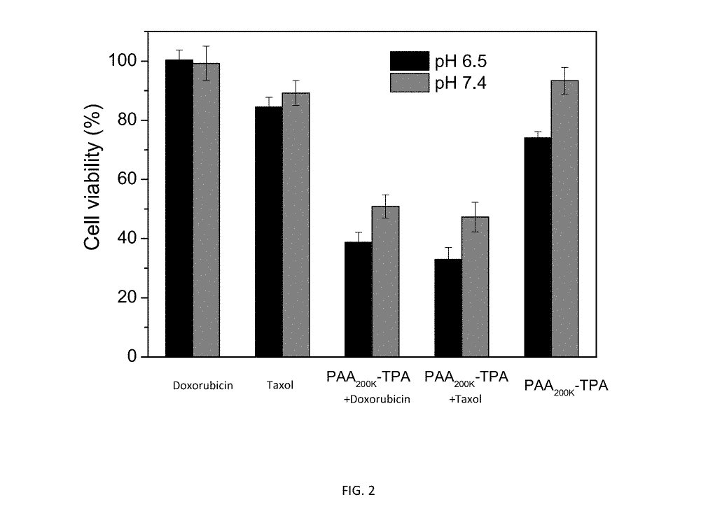

This embodiment is an in vitro cell experiment of the effect of PAA_{200K}-TPA for promoting the toxicity of different chemotherapeutic drugs on multidrug-resistant cells HepG2/ADR. According to the method described in Embodiment 5, the concentration of PAA_{200K}-TPA was 25 μg/mL, and the obtained data was shown in Table 2 and FIG. 2.

### Embodiment 10

A suspension of HepG2 cells diluted in 100 μL of a complete medium (a RPMI-1640 medium containing 10% fetal bovine serum and 1% penneillin/streptomycin) was added into a 96-well plate at a concentration of 5×10³ cells per well, cultivated at 37 degrees Celsius for 24 hours. Thereafter, the cell culture medium was aspirated, and the cells were washed twice with a PBS solution, then added respectively with a 100 μL mixed solution of a linear polyethylenimine-2,3-dimethyl maleic anhydride modifier with a molecular weight of 10,000 (LPEI_{10K}-DMA) at a concentration of 15 μg/mL and different kinds of chemotherapeutic drugs (4 μg/mL doxorubicin or 5 μg/mL taxol) as diluted in a serum-free medium at different pH (6.5, 7.4), and as a control 15 μg/mL LPEI_{10K}-DMA solution or chemotherapeutic drugs (4 μg/mL doxorubicin or 5 μg/mL taxol) were respectively added into other wells and cultured for 5 hours. The solution was then aspirated, added with 100 μL of fresh complete medium and incubated continually for 24 hours. The culture medium was aspirated, added with 804 PBS solution and 20 μL cell proliferation reagent MTS, incubated at 37 degrees Celsius for 1 hour, and then measured with a microplate reader at 490 nm for absorbance. The survival rate of non-resistant cells was calculated according to the equation ODₛₐₘₚₗₑ/OD_{blank}×100%, as shown in Table 1.

### Embodiment 11

A suspension of HepG2/ADR cells diluted in 100 IA of a complete medium (a RPMI-1640 medium containing 10% fetal bovine serum and 1% penneillin/streptomycin) was added into a 96-well plate at a concentration of 5×10³ cells per well, cultivated at 37 degrees Celsius for 24 hours. Thereafter, the cell culture medium was aspirated, and the cells were washed twice with a PBS solution, then added respectively with a 100 μL mixed solution of LPEI_{10K}-DMA at a concentration of 15 μg/mL and different kinds of chemotherapeutic drugs (4 μg/mL doxorubicin or 5 μg/mL taxol) as diluted in a serum-free medium at different pH (6.5, 7.4), and as a control 15 μg/mL LPEI_{10K}-DMA solution or chemotherapeutic drugs (4 μg/mL doxorubicin or 5 μg/mL taxol) were respectively added into other wells and cultured for 3 hours. The solution was then aspirated, added with 100 μL of fresh complete medium and incubated continually for 24 hours. The culture medium was aspirated, added with 80 μL PBS solution and 20 μL cell proliferation reagent MTS, incubated at 37 degrees Celsius for 2 hours, and then measured with a microplate reader at 490 nm for absorbance. The survival rate of multidrug-resistant cells was calculated according to the equation ODₛₐₘₚₗₑ/OD_{blank}×100%, as shown in Table 2.

### Embodiment 12

This embodiment is an in vitro cell experiment of the effect of a linear polyethylenimine-cyclohexene-1,2-dicarboxylic acid modifier with a molecular weight of 10,000 (LPEI_{10K}-TPA) for promoting the toxicity of different chemotherapeutic drugs on non-resistant HepG2 cells. According to the method described in Embodiment 10, the concentration of LPEI_{10K}-TPA was 15 μg/mL, and the obtained data was shown in Table 1.

### Embodiment 13

This embodiment is an in vitro cell experiment of the effect of LPEI_{10K}-TPA for promoting the toxicity of different chemotherapeutic drugs on multidrug-resistant cells HepG2/ADR. According to the method described in Embodiment 11, the concentration of LPEI_{10K}-TPA was 15 μg/mL, and the obtained data was shown in Table 2.

### Embodiment 14

This embodiment is an in vitro cell experiment of the effect of a branched polyethyleneimine-1-methyl maleic anhydride modifier with a molecular weight of 25,000 (BPEI_{25K}-CA) for promoting the toxicity of different chemotherapeutic drugs on non-resistant HepG2 cells. According to the method described in Embodiment 10, the concentration of BPEI_{25K}-CA was 15 μg/mL, and the obtained data was shown in Table 1.

### Embodiment 15

This embodiment is an in vitro cell experiment of the effect of BPEI_{25K}-CA for promoting the toxicity of different chemotherapeutic drugs on multidrug-resistant cells HepG2/ADR. According to the method described in Embodiment 11, the concentration of BPEI_{25K}-CA was 15 μg/mL, and the obtained data was shown in Table 2.

### Embodiment 16

This embodiment is an in vitro cell experiment of the effect of a 3rd-generation dendritic polypropyleneimine-3-carboxy-2-pentenedioic acid modifier (G3 PPI-AA) for promoting the toxicity of different chemotherapeutic drugs on non-resistant HepG2 cells. According to the method described in Embodiment 10, the concentration of G3 PPI-AA was 20 μg/mL, and the obtained data was shown in Table 1.

### Embodiment 17

This embodiment is an in vitro cell experiment of the effect of G3 PPI-AA for promoting the toxicity of different chemotherapeutic drugs on multidrug-resistant cells HepG2/ADR. According to the method described in Embodiment 11, the concentration of G3 PPI-AA was 20 μg/mL, and the obtained data was shown in Table 2.

### Embodiment 18

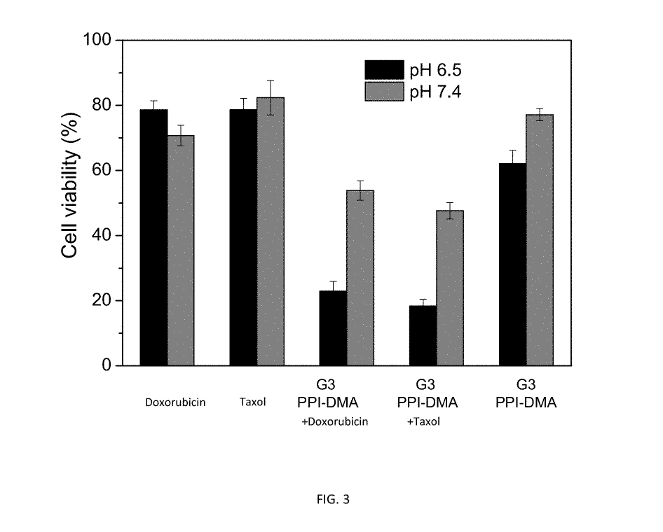

This embodiment is an in vitro cell experiment of the effect of a 3rd-generation dendritic polypropyleneimine-2,3-dimethyl maleic anhydride modifier (G3 PPI-DMA) for promoting the toxicity of different chemotherapeutic drugs on non-resistant HepG2 cells. According to the method described in Embodiment 10, the concentration of G3 PPI-DMA was 20 μg/mL, and the obtained data was shown in Table 1 and FIG. 3.

### Embodiment 19

This embodiment is an in vitro cell experiment of the effect of G3 PPI-DMA for promoting the toxicity of different chemotherapeutic drugs on multidrug-resistant cells HepG2/ADR. According to the method described in Embodiment 11, the concentration of G3 PPI-DMA was 20 μg/mL, and the obtained data was shown in Table 2.

### Embodiment 20

A suspension of HepG2 cells diluted in 100 μL of a complete medium (a RPMI-1640 medium containing 10% fetal bovine serum and 1% penneillin/streptomycin) was added into a 96-well plate at a concentration of 5×10³ cells per well, cultivated at 37 degrees Celsius for 24 hours. Thereafter, the cell culture medium was aspirated, and the cells were washed twice with a PBS solution, then added respectively with a 100 μL mixed solution of a linear polylysine-maleic anhydride modifier with a molecular weight of 2,000 (LPlys_{2K}-MA) at a concentration of 25 μg/mL and different kinds of chemotherapeutic drugs (4 μg/mL doxorubicin or 5 μg/mL taxol) as diluted in a serum-free medium at different pH (6.5, 7.4), and as a control 25 μg/mL LPlys_{2K}-MA solution or chemotherapeutic drugs (4 μg/mL doxorubicin or 5 μg/mL taxol) were respectively added into other wells and cultured for 7 hours. The solution was then aspirated, added with 100 μL of fresh complete medium and incubated continually for 24 hours. The culture medium was aspirated, added with 80 μL PBS solution and 20 μL cell proliferation reagent MTS, incubated at 37 degrees Celsius for 1 hour, and then measured with a microplate reader at 490 nm for absorbance. The survival rate of non-resistant cells was calculated according to the equation ODₛₐₘₚₗₑ/OD_{blank}×100%, as shown in Table 1.

### Embodiment 21

A suspension of HepG2/ADR cells diluted in 100 LW of a complete medium (a RPMI-1640 medium containing 10% fetal bovine serum and 1% penneillin/streptomycin) was added into a 96-well plate at a concentration of 5×10³ cells per well, cultivated at 37 degrees Celsius for 24 hours. Thereafter, the cell culture medium was aspirated, and the cells were washed twice with a PBS solution, then added respectively with a 100 μL mixed solution of LPlys_{2K}-MA at a concentration of 25 μg/mL and different kinds of chemotherapeutic drugs (4 μg/mL doxorubicin or 5 μg/mL taxol) as diluted in a serum-free medium at different pH (6.5, 7.4), and as a control 25 μg/mL LPlys_{2K}-MA solution or chemotherapeutic drugs (4 μg/mL doxorubicin or 5 μg/mL taxol) were respectively added into other wells and cultured for 7 hours. The solution was then aspirated, added with 100 μL of fresh complete medium and incubated continually for 24 hours. The culture medium was aspirated, added with 80 μL PBS solution and 20 μL cell proliferation reagent MTS, incubated at 37 degrees Celsius for 2 hours, and then measured with a microplate reader at 490 nm for absorbance. The survival rate of multidrug-resistant cells was calculated according to the equation ODₛₐₘₚₗₑ/OD_{blank}×100%, as shown in Table 2.

### Embodiment 22

This embodiment is an in vitro cell experiment of the effect of a branched polylysine-cyclohexene-1,2-dicarboxylic acid modifier having a molecular weight of 2,000 (BPLys_{2K}-TPA) for promoting the toxicity of different chemotherapeutic drugs on non-resistant HepG2 cells. According to the method described in Embodiment 20, the concentration of BPLys_{2K}-TPA was 25 μg/mL, and the obtained data was shown in Table 1.

### Embodiment 23

This embodiment is an in vitro cell experiment of the effect of BPLys_{2K}-TPA for promoting the toxicity of different chemotherapeutic drugs on multidrug-resistant cells HepG2/ADR. According to the method described in Embodiment 21, the concentration of BPLys_{2K}-TPA was 25 μg/mL, and the obtained data was shown in Table 2.

### Embodiment 24

This embodiment is an in vitro cell experiment of the effect of a 5th-generation dendritic polylysine-2,3-dimethyl maleic anhydride modifier (G5 DPlys-DMA) for promoting the toxicity of different chemotherapeutic drugs on non-resistant HepG2 cells. According to the method described in Embodiment 20, the concentration of G5 DPlys-DMA was 25 μg/mL, and the obtained data was shown in Table 1.

### Embodiment 25

This embodiment is an in vitro cell experiment of the effect of G5 DPlys-DMA for promoting the toxicity of different chemotherapeutic drugs on multidrug-resistant cells HepG2/ADR. According to the method described in Embodiment 21, the concentration of G5 DPlys-DMA was 25 μg/mL, and the obtained data was shown in Table 2.

### Embodiment 26

A suspension of HepG2 cells diluted in 100 μL of a complete medium (a RPMI-1640 medium containing 10% fetal bovine serum and 1% penneillin/streptomycin) was added into a 96-well plate at a concentration of 5×10³ cells per well, cultivated at 37 degrees Celsius for 24 hours. Thereafter, the cell culture medium was aspirated, and the cells were washed twice with a PBS solution, then added respectively with a 100 μL mixed solution of a 3rd-generation dendritic polyamidoamine-1-methyl maleic anhydride modifier (G3 PAMAM-CA) at a concentration of 50 μg/mL and different kinds of chemotherapeutic drugs (4 μg/mL doxorubicin or 5 μg/mL taxol) as diluted in a serum-free medium at different pH (6.5, 7.4), and as a control 50 μg/mL G3 PAMAM-CA solution or chemotherapeutic drugs (4 μg/mL doxorubicin or 5 μg/mL taxol) were respectively added into other wells and cultured for 3 hours. The solution was then aspirated, added with 100 μL of fresh complete medium and incubated continually for 24 hours. The culture medium was aspirated, added with 80 μL PBS solution and 20 μL cell proliferation reagent MTS, incubated at 37 degrees Celsius for 1 hour, and then measured with a microplate reader at 490 nm for absorbance. The survival rate of non-resistant cells was calculated according to the equation ODₛₐₘₚₗₑ/OD_{blank}×100%, as shown in Table 1.

### Embodiment 27

A suspension of HepG2/ADR cells diluted in 100 μL of a complete medium (a RPMI-1640 medium containing 10% fetal bovine serum and 1% penneillin/streptomycin) was added into a 96-well plate at a concentration of 5×10³ cells per well, cultivated at 37 degrees Celsius for 24 hours. Thereafter, the cell culture medium was aspirated, and the cells were washed twice with a PBS solution, then added respectively with a 100 μL mixed solution of G3 PAMAM-CA at a concentration of 50 μg/mL and different kinds of chemotherapeutic drugs (4 μg/mL doxorubicin or 5 μg/mL taxol) as diluted in a serum-free medium at different pH (6.5, 7.4), and as a control 50 μg/mL G3 PAMAM-CA solution or chemotherapeutic drugs (4 μg/mL doxorubicin or 5 μg/mL taxol) were respectively added into other wells and cultured for 3 hours. The solution was then aspirated, added with 100 μL of fresh complete medium and incubated continually for 24 hours. The culture medium was aspirated, added with 80 μL PBS solution and 20 μL cell proliferation reagent MTS, incubated at 37 degrees Celsius for 2 hours, and then measured with a microplate reader at 490 nm for absorbance. The survival rate of multidrug-resistant cells was calculated according to the equation ODₛₐₘₚₗₑ/OD_{blank}×100%, as shown in Table 2.

### Embodiment 28

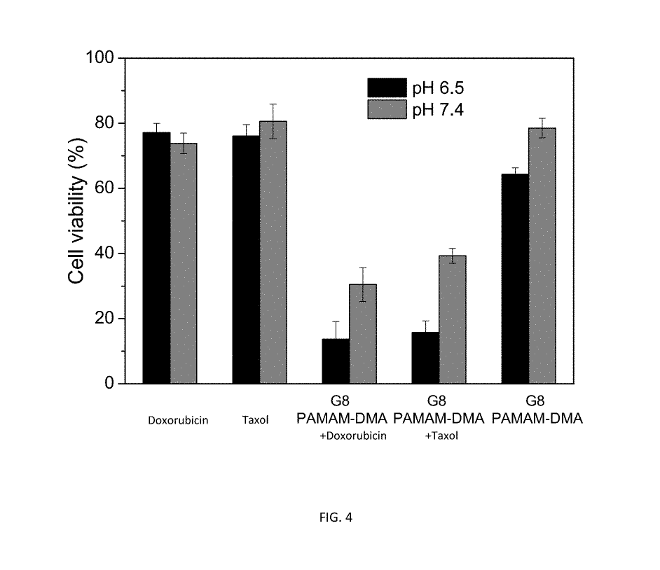

This embodiment is an in vitro cell experiment of the effect of an 8th-generation dendritic polyamidoamine-2,3-dimethyl maleic anhydride modifier (G8 PAMAM-DMA) for promoting the toxicity of different chemotherapeutic drugs on non-resistant HepG2 cells. According to the method described in Embodiment 26, the concentration of G8 PAMAM-DMA was 50 μg/mL, and the obtained data was shown in Table 1 and FIG. 4.

### Embodiment 29

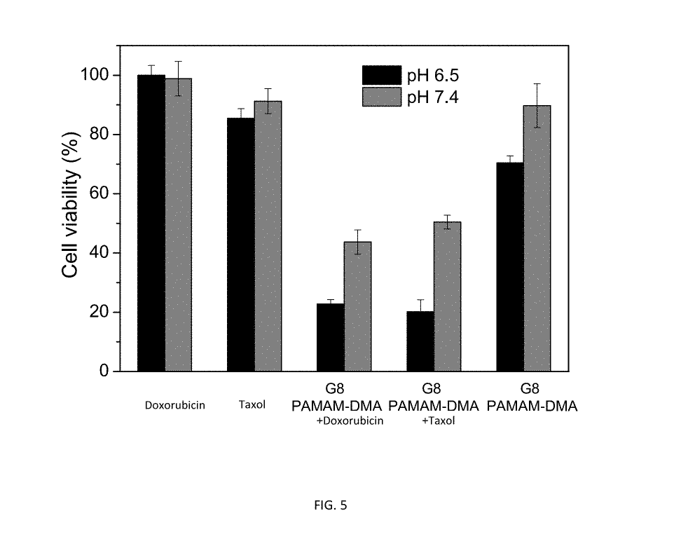

This embodiment is an in vitro cell experiment of the effect of G8 PAMAM-DMA for promoting the toxicity of different chemotherapeutic drugs on multidrug-resistant cells HepG2/ADR. According to the method described in Embodiment 27, the concentration of G8 PAMAM-DMA was 50 μg/mL, and the obtained data was shown in Table 2 and FIG. 5.

### Embodiment 30

A suspension of HepG2 cells diluted in 1004 of a complete medium (a RPMI-1640 medium containing 10% fetal bovine serum and 1% penneillin/streptomycin) was added into a 96-well plate at a concentration of 5×10³ cells per well, cultivated at 37 degrees Celsius for 24 hours. Thereafter, the cell culture medium was aspirated, and the cells were washed twice with a PBS solution, then added respectively with a 100 μL mixed solution of BPG_{50K}-DMA at a concentration of 50 μg/mL and different kinds of chemotherapeutic drugs (4 μg/mL doxorubicin or 5 μg/mL taxol) as diluted in a serum-free medium at different pH (6.5, 7.4), and as a control 50 μg/mL BPG_{50K}-DMA solution or chemotherapeutic drugs (4 μg/mL doxorubicin or 5 μg/mL taxol) were respectively added into other wells and cultured for 5 hours. The solution was then aspirated, added with 100 μL of fresh complete medium and incubated continually for 24 hours. The culture medium was aspirated, added with 80 μL PBS solution and 20 μL cell proliferation reagent MTS, incubated at 37 degrees Celsius for 1 hour, and then measured with a microplate reader at 490 nm for absorbance. The survival rate of multidrug-resistant cells was calculated according to the equation ODₛₐₘₚₗₑ/OD_{blank}×100%, as shown in Table 2.

### Embodiment 31

A suspension of HepG2/ADR cells diluted in 1004 of a complete medium (a RPMI-1640 medium containing 10% fetal bovine serum and 1% penneillin/streptomycin) was added into a 96-well plate at a concentration of 5×10³ cells per well, cultivated at 37 degrees Celsius for 24 hours. Thereafter, the cell culture medium was aspirated, and the cells were washed twice with a PBS solution, then added respectively with a 100 μL mixed solution of BPG_{50K}-DMA at a concentration of 50 μg/mL and different kinds of chemotherapeutic drugs (4 μg/mL doxorubicin or 5 μg/mL taxol) as diluted in a serum-free medium at different pH (6.5, 7.4), and as a control 50 μg/mL BPG_{50K}-DMA solution or chemotherapeutic drugs (4 μg/mL doxorubicin or 5 μg/mL taxol) were respectively added into other wells and cultured for 5 hours. The solution was then aspirated, added with 1004 of fresh complete medium and incubated continually for 24 hours. The culture medium was aspirated, added with 804 PBS solution and 204 cell proliferation reagent MTS, incubated at 37 degrees Celsius for 2 hours, and then measured with a microplate reader at 490 nm for absorbance. The survival rate of multidrug-resistant cells was calculated according to the equation ODₛₐₘₚₗₑ/OD_{blank}×100%, as shown in Table 2.

### Embodiment 32

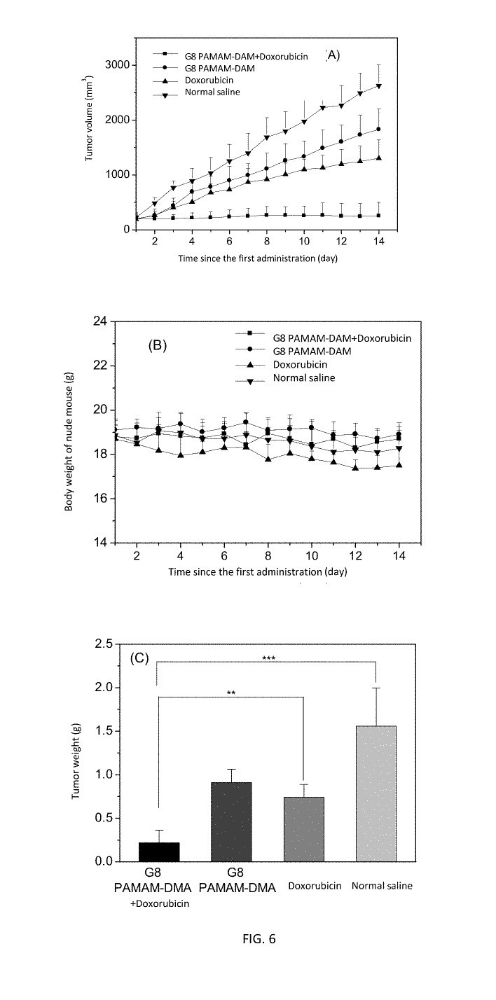

4-6-week-old female BALB/c nude mice having a weight of 18-20 g were used as experimental animals. 200 μL of a PBS solution containing 3×106 HepG2 cells was injected subcutaneously on the right side of the nude mice. The tumor volume was calculated by the equation V=a×b²/2, wherein a and b respectively represent the length and width of the tumor. When the tumor is grown to have a volume of approximately 200 mm³, the tumor-bearing mice were randomly divided into 4 groups with 5 mice per group, and were respectively subjected to intraperitoneal injection of a mixed solution of G5-DMA and doxorubicin according to a dosage of 15 mg G8 PAMAM-DMA/kg and 2 mg doxorubicin/kg. As a control, each of the remaining groups of nude mice was injected with G8 PAMAM-DMA (15 mg G8 PAMAM-DMA/kg), doxorubicin (2 mg doxorubicin/kg) and normal saline. Tumor size and body weight of nude mice were measured daily since the first administration and a second intraperitoneal injection was conducted on day 7. On day 14, all of the nude mice were sacrificed, and the dissected tumor tissues were weighed. The obtained data was shown in FIG. 6.

### Embodiment 33

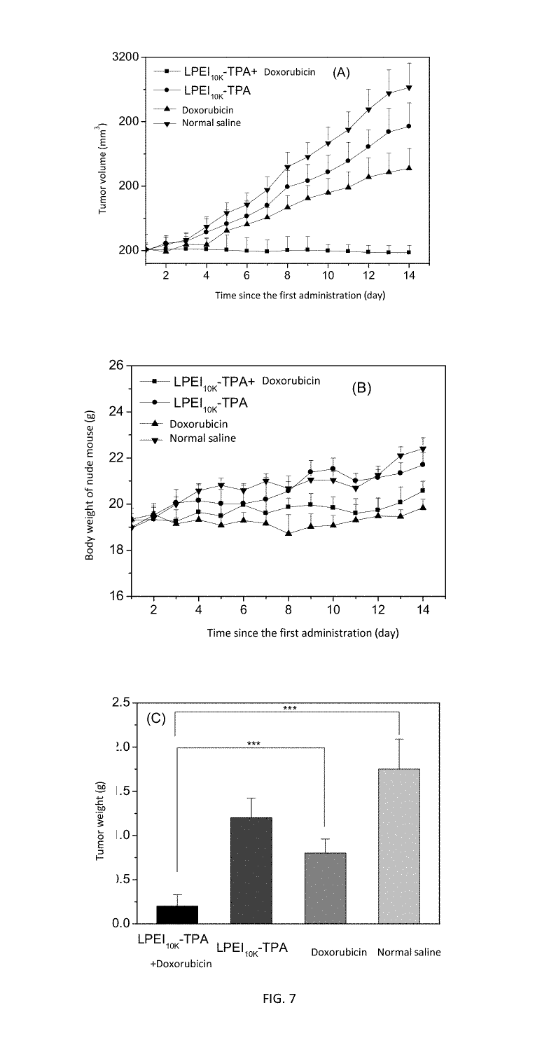

This embodiment is an experiment in which LPEI_{10K}-TPA promotes the in vivo tumor suppression effect of doxorubicin. This embodiment was conducted according to the method as described in Embodiment 32, and the dosage of the LPEI_{10K}-TPA was 10 mg/kg. The obtained data was shown in FIG. 7.

### Embodiment 34

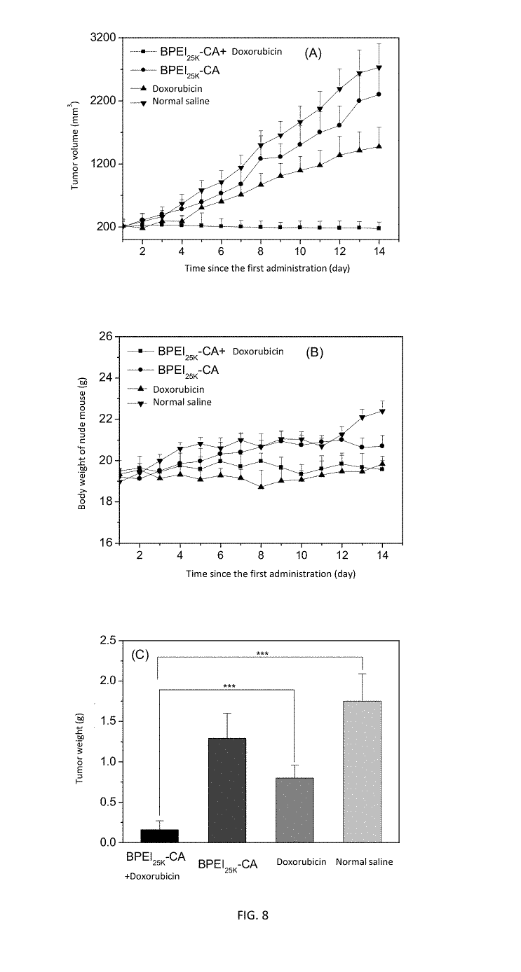

This embodiment is an experiment in which BPEI_{25K}-CA promotes the in vivo tumor suppression effect of doxorubicin. This embodiment was conducted according to the method as described in Embodiment 32, and the dosage of the BPEI_{25K}-CA was 10 mg/kg. The obtained data was shown in FIG. 8.

What is claimed is:

1. Use of a polymer as a synergist for a chemotherapeutic drug, wherein the polymer is a polymer having a 1,2-dicarboxylic acid monoamide structure, and after the polymer, a chemotherapeutic drug and a cancer cell are co-incubated in a slightly acidic environment which simulates a tumor tissue, a survival rate of the cancer cell is lower than that of a cancer cell incubated under the same conditions without the polymer; and when the polymer and the chemotherapeutic drug are injected together into a tumor-bearing animal body, the tumor-growth inhibiting effect thereof is better than that with injection of the same dosage of the chemotherapeutic drug alone.

2. The use of a polymer as a synergist for a chemotherapeutic drug according to claim 1, wherein the polymer having the 1,2-dicarboxylic acid monoamide structure is a monoamide polymer formed by a polymer containing a primary and/or secondary amine group and a 1,2-dicarboxylic acid.

3. The use of a polymer as a synergist for a chemotherapeutic drug according to claim 2, wherein the polymer containing the primary and/or secondary amine group comprises a linear polymer, a branched polymer, a hyperbranched polymer, and a dendrimer.

4. The use of a polymer as a synergist for a chemotherapeutic drug according to claim 3, wherein the linear polymer comprises polyallylamine, poly(vinyl amine), linear polyethyleneimine, polylysine and polyarginine; the branched polymer comprises branched polyethyleneimine, and high-molecular-weight polyethyleneimine obtained by crosslinking of low-molecular-weight polyethylenimine; the hyperbranched polymer comprises hyperbranched polylysine, hyperbranched polyarginine, and hyperbranched polyglycerol having a terminal amino group; and the dendrimer comprises dendritic polylysine, dendritic polyamidoamine, and dendritic polypropylenimine.

5. The use of a polymer as a synergist for a chemotherapeutic drug according to claim 3, wherein the average molecular weight of the linear polymer containing the primary and/or secondary amine group, the branched polymer and the hyperbranched polymer is ranged between 1,000 Da and 1,000,000 Da; and the generation number of the dendrimer is ranged from 2 to 10.

6. The use of a polymer as a synergist for a chemotherapeutic drug according to claim 4, wherein the average molecular weight of the linear polymer containing the primary and/or secondary amine group, the branched polymer and the hyperbranched polymer is ranged between 1,000 Da and 1,000,000 Da; and the generation number of the dendrimer is ranged from 2 to 10.

7. The use of a polymer as a synergist for a chemotherapeutic drug according to claim 2, wherein the 1,2-dicarboxylic acid comprises maleic acid, 1-methylmaleic acid, 2,3-dimethylmaleic acid, cyclohexene-1,2-dicarboxylic acid, cyclohexane-1,2-dicarboxylic acid, and aconitic acid.

8. A method of treating cancer, comprising: administering a polymer as a synergist for a chemotherapeutic drug, wherein the polymer is a polymer having a 1,2-dicarboxylic acid monoamide structure, and after the polymer, a chemotherapeutic drug and a cancer cell are co-incubated in a slightly acidic environment which simulates a tumor tissue, a survival rate of the cancer cell is lower than that of a cancer cell incubated under the same conditions without the polymer; and when the polymer and the chemotherapeutic drug are injected together into a tumor-bearing animal body, the tumor-growth inhibiting effect thereof is better than that with injection of the same dosage of the chemotherapeutic drug alone.

9. The method according to claim 8, wherein the polymer having the 1,2-dicarboxylic acid monoamide structure is a monoamide polymer formed by a polymer containing a primary and/or secondary amine group and a 1,2-dicarboxylic acid.

10. The method according to claim 9, wherein the polymer containing the primary and/or secondary amine group comprises a linear polymer, a branched polymer, a hyperbranched polymer, and a dendrimer.

11. The method according to claim 10, wherein the linear polymer comprises polyallylamine, poly(vinyl amine), linear polyethyleneimine, polylysine and polyarginine; the branched polymer comprises branched polyethyleneimine, and high-molecular-weight polyethyleneimine obtained by crosslinking of low-molecular-weight polyethylenimine; the hyperbranched polymer comprises hyperbranched polylysine, hyperbranched polyarginine, and hyperbranched polyglycerol having a terminal amino group; and the dendrimer comprises dendritic polylysine, dendritic polyamidoamine, and dendritic polypropylenimine.

12. The method according to claim 10, wherein the average molecular weight of the linear polymer containing the primary and/or secondary amine group, the branched polymer and the hyperbranched polymer is ranged between 1,000 Da and 1,000,000 Da; and the generation number of the dendrimer is ranged from 2 to 10.

13. The method according to claim 11, wherein the average molecular weight of the linear polymer containing the primary and/or secondary amine group, the branched polymer and the hyperbranched polymer is ranged between 1,000 Da and 1,000,000 Da; and the generation number of the dendrimer is ranged from 2 to 10.

14. The method according to claim 9, wherein the 1,2-dicarboxylic acid comprises maleic acid, 1-methylmaleic acid, 2,3-dimethylmaleic acid, cyclohexene-1,2-dicarboxylic acid, cyclohexane-1,2-dicarboxylic acid, and aconitic acid.
